(19) United States
(12) Patent Application Publication
(10) Pub. No.: US 2021/0346672 A1
(43) Pub. Date: Nov. 11, 2021

(54) CAPPING AND CLEANSING DEVICES FOR THREADED VASCULAR ACCESS CONNECTORS

(71) Applicant: CleanSite Medical, Inc., Solana Beach, CA (US)

(72) Inventors: John Grant, Solana Beach, CA (US); Richard A. Pluth, San Diego, CA (US); Daniel M. Chambers, Solana Beach, CA (US)

(73) Assignee: CleanSite Medical, Inc., Solana Beach, CA (US)

(21) Appl. No.: 17/379,988

(22) Filed: Jul. 19, 2021

Related U.S. Application Data
(63) Continuation-in-part of application No. 17/342,630, filed on Jun. 9, 2021.
Application No. 17/342,630 is a continuation of application No. 16/795,565, filed on Feb. 19, 2020, now Pat. No. 11,065,431.
Application No. 16/795,565 is a continuation-in-part of application No. 16/059,029, filed on Aug. 8, 2018, now abandoned.
(60) Provisional application No. 63/053,703, filed on Jul. 19, 2020.
(60) Provisional application No. 62/807,239, filed on Feb. 19, 2019.
(60) Provisional application No. 62/542,770, filed on Aug. 8, 2017.

(51) Int. Cl. A61M 39/16; A61M 39/20; A61M 39/18

(52) U.S. Cl. CPC A61M 39/162; A61M 2205/0205; A61M 39/18; A61M 39/20

(57) ABSTRACT

Capping and cleansing devices for capping and cleansing threaded vascular access connectors, particularly luer access devices such as needlefree vascular access connectors, threaded male luer connectors, and threaded open female luer connectors, and methods for using such devices, are described. The devices of the invention include interconnected inner and outer housings that a user can transition between a locked or engaged position to allow them to rotate in unison and a unlocked or disengaged position that allows the outer housing to rotate independently of the inner housing about the device's central axis, and a compressible cleansing matrix secured in the device (preferably in a well in the outer housing).

### RELATED APPLICATIONS

This application claims the benefit of and priority to, commonly owned, co-pending U.S. provisional patent application No. 63/053,703, filed on 19 Jul. 2020 (Docket No. CSM-0022ALL-PV), and is a continuation-in-part of U.S. patent application Ser. No. 17/342,630, filed on 9 Jun. 2021 (Docket No. CSM-0022-CT), which is a continuation of allowed U.S. patent application Ser. No. 16/795,565, filed on 19 Feb. 2020 (Docket No. CSM-0022-UT), which claims the benefit of and priority to (now-expired) U.S. provisional patent application No. 62/807,239, filed on 19 Feb. 2019 (Docket No. CSM-0022-PV) and U.S. patent application Ser. No. 16/059,029, filed on 8 Aug. 2018 (Docket No. CSM-0021-UT) (to which U.S. Ser. No. 16/795,565 (Docket No. CSM-0022-UT) is a continuation-in-part), which claims the benefit of and priority to (now-expired) U.S. provisional patent application No. 62/542,770, filed on 8 Aug. 2017 (Docket No. CSM-0021-PV). All of the aforementioned priority applications are hereby incorporated by reference, each in its entirety for any and all purposes.

### TECHNICAL FIELD OF THE INVENTION

This invention is directed to cleansing devices for cleansing and capping medical devices, particularly luer access devices such as needlefree, valved connectors (NCs), threaded male luer connectors, and threaded open female luer connectors, and methods for making and using such articles.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

Needlefree vascular access systems and devices have become commonplace in medicine due to occupational health and safety regulations designed reduce the risk to health care workers from needle stick and similar injuries. Today, these systems and devices include IV administration and extension sets, catheters (including peripheral IV catheters, central lines (including peripherally inserted central lines), hemodialysis catheters, etc.), and medical valves (also referred to herein as “needlefree connectors” or “NFCs”). However, the introduction of these systems and devices has resulted in increases in “nosocomial infection”, i.e., infections that originate from or occur in a hospital or hospital-like setting. In the U.S., nosocomial infections are estimated to occur in at least 5% of all acute care hospitalizations. The estimated incidence is more than 2,000,000 cases per year, resulting in significant morbidity, mortality, and an expense. Indeed, nosocomial infections are estimated to more than double the mortality and morbidity risks of any admitted patient, and likely result in about 100,000 deaths a year in the United States alone. Similar problems occur with patients receiving care in non-acute settings, as well. Common sites for the transmission of contaminating microorganisms into a patient's bloodstream are found on medical devices such as luer access devices, vials, needlefree (or needle free) connectors (NFCs) or needlefree valves, and the injection ports of vessels, tubing, and catheters. The incidence of such infections in patients is increasing, and infection control practitioners (ICPs) often cite improper cleansing of sites as a major source of such infections.

As described above, the widespread use of needlefree vascular access systems and devices in medical settings has contributed to a marked increase in the incidence of hospital-acquired infections (HAIs), particularly blood stream infections (BSIs). To reduce the risk of infection from a needlefree vascular access systems and devices contaminated with potentially pathogenic microorganisms, standard practice today requires that a nurse or other healthcare worker clean (or “scrub”) the surface of such devices that are in the fluid path prior to access. This is supposed to be accomplished by vigorously rubbing the exterior surfaces in the fluid path with a sterile alcohol swab or wipe immediately prior to making a fluid connection to the particular device (typically the threaded valve portion of a needlefree connector or the threaded portion of a male or female catheter hub), for example, by attaching a syringe to a NFC's threaded valve portion to deliver a medication to an IV catheter already connected to the patient. Given the magnitude of the mortality and morbidity associated with HAIs, particularly with regard to central line-associated blood stream infections (CLABSIs), and the large number of blood stream infections that result from peripheral intravenous catheters (PIVCs) and central lines (so-called “peripheral line-associated blood stream infections (PLABSIs) and central line-associated blood stream infections (CLABSIs), respectively), a long-recognized yet significant unmet need exists for articles or devices that can be used to reduce or eliminate the risk of initiating an HAI merely by accessing a patient's vasculature through a needlefree system providing access to a blood vessel of a patient.

Traditionally, and as noted above, cleaning, cleansing, or disinfecting a potentially contaminated surface of a component of a needlefree access system involved a protocol of alcohol swabbing prior to making the necessary connections to the site. Alcohol swabs are typically small pads of cotton gauze soaked in isopropyl alcohol (IPA), packed individually in foil packages to prevent evaporation of the IPA from the swab prior to use. Properly used, the package is opened at or near the site to be swabbed. With gloved hands, the swab is removed by a nurse or other healthcare provider and used to scrub the top and side surfaces of the valve portion of the NFC to be connected. After use, the swab and foil package are discarded and the cleansed valve portion of the NFC is allowed to dry, usually 20-30 seconds, immediately prior to making any connection. This “drying” period is important because, as the IPA dries, it breaks open the cellular walls of microorganisms, thereby killing them.

Unfortunately, because of increased duties and responsibilities, shrinking nursing staffs, and inadequate training, alcohol swabbing (or scrubbing) is often not performed or is poorly executed. A poorly swabbed site can carry microorganisms that, if allowed to enter a patient's body, can cause serious, and potentially life-threatening, infection. In addition, supervisory oversight is nearly impossible, because unless a supervisor actually observes swabbing as it is performed, the supervisor cannot know whether or not the scrubbing procedure was done properly or performed at all. Indeed, reported compliance with such “scrub the hub” protocols has been as low as 10%. Further, without at least a sufficient microscopic examination for microbial residue (e.g., biofilm), there may be no evidence of “scrubbing the hub” being performed.

Thus, a significant need still exists for devices and techniques cleanse sites on medical devices prior to their use with or connection to patients, and which eliminate technique-related and training issues and provide an unequivocal indicator that a site is clean prior to accessing a patient's vascular system.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “species”, when used in the context of describing a particular compound or molecule species, refers to a population of chemically indistinct molecules.

### SUMMARY OF THE INVENTION

The object of the invention is to address these long-standing but still unmet needs. This invention addresses these needs by providing patentable, single-use cleansing (disinfecting) and capping devices or articles that can be used to effectively and efficiently cleanse/disinfect and cap, and preferably sterilize, exposed surfaces of medical articles such as luer access devices, particularly needlefree connectors, particularly the accessible surface(s) of the threaded valve portions of needlefree connectors and catheter hubs, particularly those surfaces (valve surfaces, threads, male luer tapers, open female luer hubs, etc.) that may become contaminated with pathogens or other infectious reagents and which form part of the fluid communication pathway between an external fluid source (e.g., a medicine-filled syringe with a male luer fitting, an IV bag, a dialysis machine, etc.) and a patient's blood stream. In the context of the invention, “cleanse” encompasses cleaning, disinfecting, sanitizing, and/or sterilizing, whereas “capping” refers to using a device, i.e., a “cap”, to cover a surface, or set of surfaces, of an NFC or catheter hub so as to limit or prevent exposure of their exterior surface(s) to the environment (e.g., the air circulating in a hospital's intensive care unit(s) or other wards, the microbiome resident on a patient's skin, clothing, bedding, unclean fingers, etc.) for a period longer than necessary to cleanse the desired surface(s) of a needlefree connector.

Thus, in one aspect, the invention provides capping and disinfecting devices for medical devices such as luer access devices or threaded vascular access connectors, including needlefree, valved vascular access connectors (NFCs). In general, such devices include an inner housing configured to allow the device to be screwed onto and unscrewed from the threaded valve portion of the vascular access device, an outer housing that rotatably retains the inner housing but which a user can, when desired, rotate independently of the inner housing to provide scrubbing or disinfecting action, and a compressible cleansing matrix preferably impregnated with a disinfectant, for example, a 70% IPA solution. The device also preferably includes an easily removable seal to maintain sterility and prevent loss of the disinfectant after the device is assembled until such time as it is used in the field to outer housing and cleanse an NFC.

Other aspects of the invention concern active methods of cleansing and/or capping vascular access connectors using a capping and cleansing device according to the invention. In addition to methods for cleansing accessible surfaces of vascular access connectors and the like, the devices of the invention provide methods of reducing infection risk in a patient connected to devices such as a peripheral IV line, a central IV line, a peripherally inserted central catheter, hemodialysis catheter, or other fluid line configured for pumped or gravity-fed delivery of fluids directly into the patient's vasculature.

Features and advantages of the invention will be apparent from the following detailed description, and appended claims.

### BRIEF DESCRIPTION OF THE DRAWINGS

These and other aspects will now be described in detail with reference to the following drawings. Unless otherwise indicated, it is understood that the drawings are not to scale, as they are intended merely to facilitate understanding of the invention as opposed to specific dimensions, etc. In the drawings, like numbers in two or more drawings represent like elements.

FIG. 1 shows an exploded cutaway view of a representative example of a capping and cleansing device of the invention designed to be threaded onto a threaded open female luer connector, as well as a cutaway view of such a device threaded onto such a connector.

FIG. 2 shows an exploded cutaway view of a representative example of a capping and cleansing device of the invention designed to be threaded onto a threaded male luer connector, as well as a cutaway view of such a device threaded onto such a connector.

The details of one or more embodiments are set forth in the accompanying drawings and the description below of various representative embodiments. Other features and advantages will be apparent from the description and drawings, and from the claims.

### DETAILED DESCRIPTION

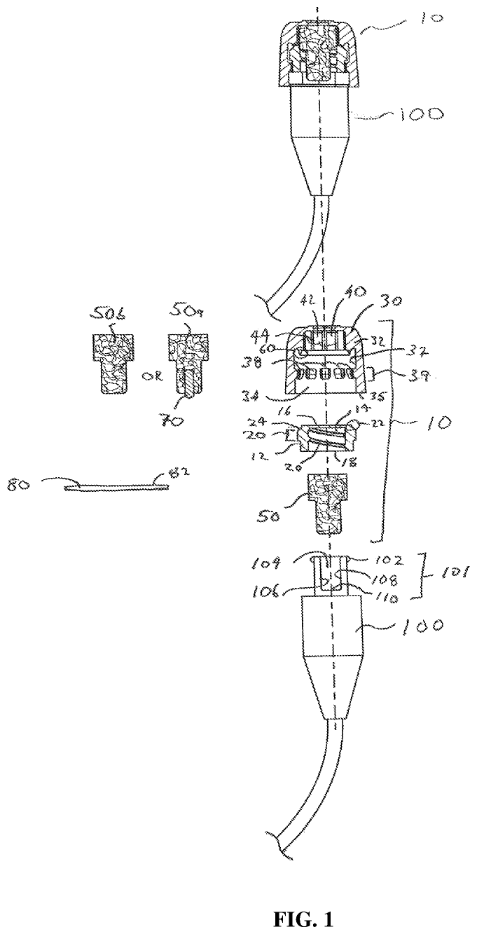

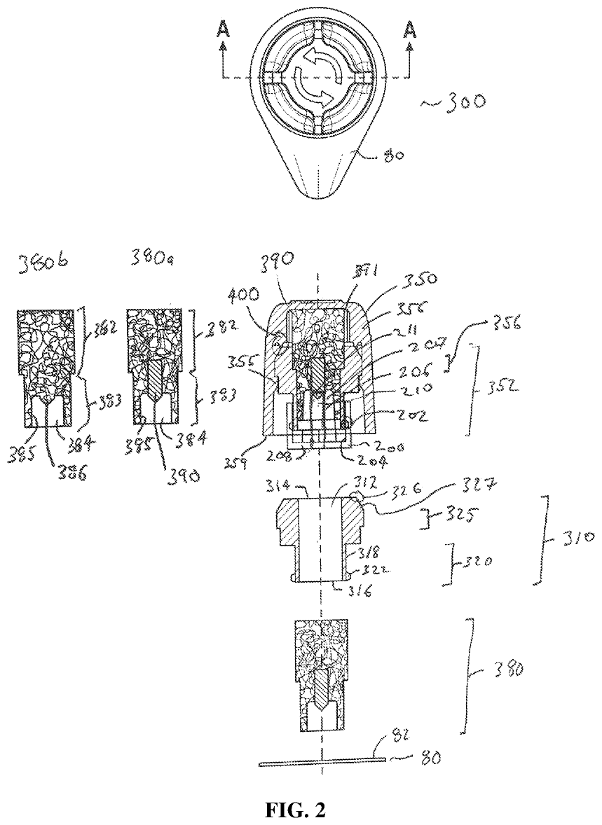

In the following detailed description, reference is made to the accompanying figures (FIGS. 1 and 2), which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here.

As described, this invention provides patentable, single-use cleansing (disinfecting) and capping devices or articles that can be used to effectively and efficiently cleanse/disinfect and cap, and preferably sterilize, exposed surfaces of medical articles such as vascular access connectors, particularly those that have luer connectors, for example, needlefree connectors, male luer connectors, open female luer connectors, and the like, especially the accessible surface(s) of the threaded valve portions of such connectors (e.g., catheter hubs having luer connectors), particularly those surfaces (valve surfaces, threads, male luer tapers, open female luer hubs, etc.) that may become contaminated with pathogens or other infectious reagents and which form part of the fluid communication pathway between an external fluid source (e.g., a medicine-filled syringe with a male luer fitting, an IV bag, a dialysis machine, etc.) and a patient's vasculature.

Accordingly, in one aspect, the invention provides capping and disinfecting devices for medical devices such as luer access devices or threaded vascular access connectors, including needlefree, valved vascular access connectors (NFCs). In general, such devices include an inner housing configured to allow the device to be screwed onto and unscrewed from the threaded valve portion of the vascular access device, an outer housing that rotatably retains the inner housing but which a user can, when desired, rotate independently of the inner housing to provide scrubbing or disinfecting action, and a compressible cleansing matrix preferably impregnated with a disinfectant, for example, a 70% IPA solution. The device also preferably includes an easily removable seal to maintain sterility and prevent loss of the disinfectant after the device is assembled until such time as it is used in the field to outer housing and cleanse an NFC.

More particularly, the devices of the invention include an inner housing. In some preferred embodiments, the inner housing is comprised of a sidewall that bounds a central, interior (preferably cylindrical) bore which spans between oppositely disposed first and second (or upper and lower, respectively) openings. In many of these embodiments, the first (upper) opening is sized to allow the compressible cleansing matrix resident at least in part in a matrix well or otherwise attached to the inner surface of the outer housing to protrude into and through the opening into the inner housing's central bore so that the compressible cleansing matrix can engage one or more exterior surfaces of a luer access device (e.g., a needlefree connector or catheter hub) when the capping and cleaning device is secured to the luer access device. The second (lower) opening of the inner housing is sized to allow at least a portion of the threaded vascular access connector, e.g., the threaded portion of a needleless (or needlefree) connector, the male luer taper of a threaded male luer connector of a syringe, the threaded female luer connector of a central line catheter hub (e.g., a catheter hub of a hemodialysis catheter), etc., to be capped and/or cleansed to be inserted into capping and cleansing device of the invention.

In some embodiments, the interior wall (or bore-facing surface) of the inner housing's central bore includes one or more thread-engaging tabs (or threads), preferably two (or more) oppositely disposed (or otherwise spaced) thread-engaging tabs, preferably near the lower opening of the central bore. In these embodiments, the thread-engaging tab(s) (or threads) is(are) configured to engage a complementary threaded region (or thread tab-containing region) on the exterior surface of, for example, a needlefree connector (or open female luer connector) such that the capping and cleansing device can, via association of the thread tabs or threads on the interior of the inner housing's central bore with complementary threads (or thread tabs) on the threaded portion of the threaded vascular access connector, be securely threaded onto (or otherwise removably connected with) the targeted threaded portion of a threaded vascular access connector (e.g., the threaded valve portion of a needlefree connector for capping and, if desired, cleansing the NFC's valve portion).

Other embodiments of this aspect provide for connection to a threaded vascular access connector having a threaded male luer connector. In these embodiments, the inner housing preferably includes threads or thread tabs configured to engage a complementary threaded region of the threaded vascular access connector. Typically, the threads or thread tabs of the inner housing will be disposed on a portion of an outer surface of the inner housing, thereby allowing them to engage with complementary threads on the inner (male luer taper-facing) surface of the outer cylindrical wall of a male luer connector, as is found, for example, at the distal (patient-proximate) end of many widely used IV administration or extension sets.

In some preferred embodiments, the outer surface of the inner housing includes an outer housing-retaining region that includes one or more structures, for example, a circumferential flange (or spaced flange elements), that allow the inner housing to be retained in the outer housing via association with one or more complementary structures (e.g., a circumferential flange (or spaced flange elements) or other suitable engaging elements) on the inner surface of the sidewall of the outer housing after the inner and outer housings are assembled during manufacturing into a functional capping and cleansing device according to the invention. Preferably, such configurations of complementary retaining elements also allow for smooth, low friction movement (i.e., rotation) of the inner and outer housings in relation to each other during certain operations, for example, during a disinfection procedure of a needlefree connector, male luer fitting, or open female luer connector (e.g., as often found on central line catheter hubs, stopcocks, hemodialysis catheter hubs, etc.). In some of these embodiments, the retaining element(s) of the inner housing can mechanically engage an adjacent region on the inner surface of the sidewall of the outer housing, for example, when a user squeezes or otherwise applies sufficient force to the outer housing to deform it so as to allow engaging regions on the inner surface of the outer housing to engage corresponding engaging regions on the exterior surface of the inner housing so as to allow the inner and outer housings to rotate in unison (as would occur, for example, when a user attaches or removes the capping/cleansing device from an NFC). In some of these embodiments, the retaining element(s) of the inner housing can also serve as engaging elements with complementary regions, features, or structures on the inner surface of the sidewall of the outer housing adjacent or otherwise in close proximity thereto. In other embodiments, the outer surface of the inner housing further includes one or more outer housing engaging elements or regions designed to associate with one or more inner housing engaging elements or regions disposed on the interior or inner surface of the outer housing. Examples of such elements include, for example, a circumferential band of spaced teeth or teeth-like elements protruding from the exterior surface of the inner housing and positioned below the outer housing-retaining region (e.g., a circumferential flange), which teeth (or other suitable engaging structures) can be engaged by complementary structures arrayed on the interior surface of the outer housing when the housings are assembled into a functional subassembly.

In other preferred embodiments, the upper exterior surface of the inner housing includes an outer housing-engaging region that includes one or more structures that allow the inner housing to mechanically engage complementary structures (e.g., pawls or other suitable engaging elements) on the inner surface of the top of the outer housing so that when the complementary engaging elements of the outer housing and inner housing are brought into close proximity the engaging elements of the outer housing and inner housing engage, allowing the outer housing and inner housing to rotate in unison. Certain preferred embodiments of outer housing-engaging structures include spaced teeth (or other suitable engaging elements) arrayed on the top or upper surface of the inner housing's preferably cylindrical sidewall. As will be appreciated, when such inner and outer housing engaging elements are unmated or disengaged, a user can rotate or spin the outer housing in relation to the inner housing, as is, for example, done during a cleansing or disinfecting operation of the needlefree connector to which the device of the invention is attached. Thus, when the capping and cleansing device is secured to a needlefree connector, when such engaging elements are not functionally associated (or mated or otherwise engaged), a user can rotate the outer housing (and compressible cleansing matrix) in relation to the inner housing and connected needlefree connector. On the other hand, when the complementary elements on the inner surface of the top of the outer housing and the upper surface of the top of the inner housing are engaged (in whole or even partially), such as when a user applies downward pressure to the device to place it on or remove it from a needlefree connector, the inner and outer housings rotate together, allowing, for example, the capping and cleansing device to be attached to or removed from the NFC.

In some preferred embodiments, the inner housing also includes a sealing member configured to provide a fluid tight seal between the capping and cleansing device of the invention and a threaded vascular access connector connected thereto. In some embodiments the sealing member is an O-ring (or comparable seal) preferably disposed in a channel formed in the inner surface of the wall of the inner housing proximate to the second (lower) opening, typically below the thread-engaging tab(s) (or threads).

The devices of the invention also include an outer housing adapted or configured to retain the inner housing therein after functional device assembly such that, when the capping and cleansing device is attached to a threaded vascular access connector, under certain conditions the outer housing can rotate (preferably about its central axis) in relation to the inner housing (which inner housing is secured to the vascular access connector). Any suitable configuration of complementary mechanical or structural features or elements on facing or opposing surfaces can be used to provide retention of the inner housing inside the outer housing's main cavity and to allow for engagement and disengagement of the outer housing from the inner housing in order to allow the outer housing to be rotated in relation to the inner housing when the device is attached to a threaded vascular access connector (e.g., a needlefree connector, male luer, or open female luer) and a user desires to cleanse the corresponding surface(s) of the threaded vascular access connector (e.g., NFC) using the capping and cleansing device of the invention.

In some embodiments, when the device of the invention is attached to a threaded vascular access connector, the inner and outer housings adopt a disengaged, neutral, or rotating configuration relative to each other such that a user can rotate the outer housing in relation to the inner housing to perform a cleansing operation on the valve portion of the threaded vascular access connector to which the capping and cleansing device of the invention is attached. Such a disengaged, neutral, or rotating configuration can be achieved by any suitable approach, including by providing complementary engaging elements or structures on adjacent surfaces of the inner and outer housings that under certain conditions, for example, when the outer housing is pulled up, pushed down, or squeezed by a user in relation to the inner housing, engage each other; otherwise the engaging elements remain disengaged, which allows rotation of the outer housing in relation to the inner housing when the device is secured to a threaded vascular access connector (e.g., an NFC, male luer, or open female luer). Features that allow transitioning between engaged and disengaged positions include springs or biasing or resilient elements or materials. In other embodiments, when the capping and cleansing device of the invention is attached to a threaded vascular access connector (e.g., a needlefree connector, a male luer connector, an open female luer connector, etc.), the inner and outer housings adopt an engaged configuration relative to each other such that they rotate in unison (typically concentrically, each about its central axis) unless a user applies sufficient force to the outer housing to cause the engaging elements to disengage and thus allow the outer housing to be rotated independently of the inner housing. Rotation is preferably allowed to occur in both directions, i.e., clockwise and anti- or counterclockwise, without unthreading the capping and cleansing device of the invention from the vascular access connector.

The outer housing includes a cavity, preferably a cylindrical cavity, designed to receive and retain the inner housing using one or more features or elements that allow the outer housing to be rotated in relation to the inner housing if and when desired. The cavity is preferably formed by a curved outer sidewall that in some embodiments is joined (preferably, integrally) to a top portion of the housing about its periphery and which also preferably has a concentric central matrix well or matrix attachment region to or with which the compressible cleansing matrix is attached or otherwise associated, although in some embodiments some degree of eccentricity between the matrix well and central rotational axis of the outer housing may be desired. In some embodiments, the outer housing is formed by a sidewall that is tapered and/or has one or more steps.

In some preferred embodiments, the inner surface of the top of the outer housing includes one or more inner housing engaging elements or structures (e.g., teeth) designed to releasably engage complementary structures in the outer housing-engaging region on the top of the inner housing. Engagement of the outer housing's inner housing engaging structure(s) with those in the outer housing-engaging region of the inner housing allow a user to rotate the outer housing and inner housing in unison, for example, as a capping and cleansing device's inner housing is screwed onto the threaded portion of a needlefree connector to be cleansed and/or capped. Once the device is releasably secured to a needlefree connector via the inner housing, the outer housing's inner housing engaging elements or structure(s) can be (or are) disengaged from the outer housing-engaging elements of the inner housing, for example, by the biasing action or resilience of the compressible cleansing matrix, thereby allowing a user to rotate the outer housing about its central axis in relation to the inner housing. A representative example of such engaging structures is shown in commonly owned U.S. non-provisional patent application Ser. No. 16/795,565, although features such as an inner housing having an opening in its top to allow a compressible cleansing matrix attached to the inner surface of the top of the outer housing to extend into the bore of the inner housing so that it can contact surfaces of a vascular access connector upon connection of the former to the latter are also envisioned.

In some of these embodiments, the outer housing may include one or more vents to allow fluid and/or air from inside the device to escape as the capping and cleansing device is secured to a needlefree connector, while in other embodiments, no vent(s) is(are) provided. In embodiments with one or more vents, a membrane, filter, or other permeable or semi-permeable barrier may be employed to allow a unidirectional or bidirectional flow of air, gas, or vapor through the vent(s) but prevent the movement of microorganisms (e.g., bacteria, fungi, viruses, etc.) into the capping and cleansing device of the invention.

In certain preferred embodiments, the outer surface of the outer housing of a capping and cleansing device according to the invention includes one or more grip-enhancing structures (e.g., a plurality of vertical ridges) or coatings. Such grip-enhancing structures or coatings facilitate a user's grasp of the housing of a capping and cleansing device between her/his fingers, which can be helpful not only during insertion and removal of a needlefree connector from the capping cleansing device, but also during the cleansing process, where the user rotates the outer housing in relation to the inner housing in order to scrub and thereby clean or cleanse the surface(s) of the inserted needlefree connector with the compressible cleansing matrix of the device.

In some preferred embodiments, the devices of the invention include one or more elements or features arrayed on facing surfaces of the inner and outer housings that allow a user to sense that the outer housing is rotating in relation to the inner housing in order to provide cleansing action on the valve surface of the NFC to which the device is attached. Such sensory feedback can include one or more of auditory, tactile, and/or visual stimuli generated from the device by rotation of the outer housing in relation to the inner housing.

In the devices of the invention, the inner and outer housings are manufactured separately by any suitable process, for example, 3D-printing, injection molding, etc., and then assembled into a two-part subassembly in which the inner housing is retained within the main cavity of the outer housing by one or more complementary retaining elements, features, or structures on each housing. The inner and outer housings also include complementary mechanical or structural engaging elements, features, or structures on one or more interfacing surfaces that can be engaged and disengaged so as to allow the inner and outer housings to rotate together or to allow the outer housing to rotate independently of the inner housing. In this way, the inner and outer housings can be associated such that they can rotate in unison, allowing a user to thread (or screw) the device onto or remove (unscrew) it from the threaded valve portion of threaded vascular access connector (e.g., an NFC) if and when desired, while also making it possible for a user to rotate the outer housing in relation to the inner housing, thereby allowing the compressible cleansing matrix to effectively scrub or cleanse the region(s) of a threaded valve portion of an NFC to which it is attached. In certain preferred embodiments, the inner and outer housings further include complementary mechanical or structural housing sealing elements, features, or structures on one or more interfacing surfaces that allow formation of seal between adjacent surfaces of the inner and outer housings, which seal is preferably substantially fluid tight but does not substantially hinder or inhibit rotation of the outer housing in relation to the inner housing during performance of a cleansing procedure or process by a user. In some embodiments, the inner housing may also include a seal that interacts with the needlefree connector to form an additional or alternate seal.

A capping and cleansing device of the invention also includes a compressible cleansing matrix disposed therein. In most embodiments, the compressible cleansing matrix is disposed in a matrix well or the like in the interior of the outer housing, although any suitable retaining configuration can be employed that allows the compressible cleansing matrix to rotate in conjunction with rotation of the outer housing so as to provide the capability of using the compressible cleansing matrix to scrub or otherwise clean, cleanse, or disinfect the surface(s) of the valve region of a threaded vascular access connector (e.g., a needlefree connector, a male luer fitting, an open female luer connector, etc.). As will be appreciated, the compressible cleansing matrix is positioned to contact one or more exterior surface(s) of a threaded vascular access connector connected to the capping and cleansing device. The compressible cleansing matrix, for example, an open-cell or felted foam, is preferably retained in the matrix well by one or more matrix retaining elements, which element(s) assist in retention of the compressible cleansing matrix in the matrix well in addition to transmission of rotational forces from the outer housing to the compressible cleansing matrix as occurs during a procedure to disinfect or cleanse a threaded vascular access connector (e.g., a needlefree connector, male luer, or open female luer). As will be appreciated, because the compressible cleansing matrix is retained in (and those rotationally associated with) the matrix well of the outer during, such rotation (of the outer housing and compressible cleansing matrix), the compressible cleansing matrix also rotates in relation to the inner housing when the outer housing is rotated during a procedure to disinfect or cleanse the threaded vascular access connector to which the capping and cleansing device is connected. The compressible cleansing matrix attached to or otherwise associated with the outer housing can be axially compressed (i.e., compressed along the central axis of the outer housing's matrix well) upon insertion of a threaded vascular access connector into such a capping and cleansing device. In some embodiments, the compressible cleansing matrix serves as a spring or resilient member that can cause the outer housing to move upward in relation to the inner housing when a device according to the invention is connected to a vascular access device.

Because the surface(s) of the threaded vascular access connector to be cleansed may be contaminated with microorganisms that form a biofilm (i.e., a matrix of microorganisms and extracellular material attached to a surface, which enables the microorganisms, typically bacteria and/or fungi, to adhere to a surface and carry out certain biochemical processes), the compressible cleansing matrix also preferably has sufficient mechanical integrity when compressed and rotated to allow it to disrupt any biofilm that may be present on the surface of the threaded vascular access connector to be cleansed, as can occur by rotating, twisting, or otherwise moving the then-compressed cleansing matrix in relation to the threaded vascular access connector, for example, by rotating the outer housing (to which the compressible cleansing matrix is attached) in relation to the inner housing of the capping and cleansing device and the threaded vascular access connector to which inner housing is releasably attached. The resulting friction between the compressed cleansing matrix and surface of the threaded vascular access connector disrupts the biofilm, thereby cleansing, and preferably sterilizing, that/those surfaces of the threaded vascular access connector. Leaving the capping and cleansing device secured to (i.e., capping) the threaded vascular access connector after such a cleansing operation will limit, and preferably preclude, biofilm regrowth and/or the microbial recolonization of cleansed surfaces (which remain in contact with the compressible cleansing matrix).

In preferred embodiments, the compressible cleansing matrix includes one or more cleansing reagent species dispersed therein, preferably at the time the device is manufactured, although in some embodiments, the cleansing reagent may be dispersed into the matrix just prior to the matrix coming into contact with a needlefree connector. In embodiments of the latter sort, the cleansing reagent is preferably housed in the housing of the capping and cleansing device in a reservoir configured to be ruptured just prior to performance of a cleansing operation. In some embodiments, the capping and cleansing device of the invention will include a valve or opening to allow liquid in the cleansing reagent to evaporate.

In some preferred embodiments, the compressible cleansing matrix includes two or more components. In some of such embodiments, one component of the matrix is attached to the inner surface of the outer housing and another component is secured to the inner surface of the wall forming the inner housing, preferably between protruding threaded regions adapted to engage complementary threads on a needlefree connector. If present, the component of the compressible cleansing matrix secured to the inner surface of the inner housing wall is preferably configured to radially compress upon association with a needlefree connector to be capped and cleansed.

In preferred embodiments, the compressible cleansing matrix is formed such that those portions intended to contact potentially contaminated surfaces of a threaded vascular access connector to which the device of the invention is attached have a shape that is complementary to the surfaces to be cleansed. For example, in some embodiments the cleansing matrix is shaped to have a tapered cavity configured to conform to the exterior shape of at least a portion of a male luer taper, thereby optimizing the cleansing association between the compressible cleansing matrix and male taper when the capping and cleansing device of the invention is connected to, for example, a hub having a male luer connector at the distal end of an IV set.

In some embodiments, the compressible cleansing matrix is assembled with the outer housing prior to assembly of the inner and outer housings. In other embodiments, the compressible cleansing matrix is assembled with the outer housing after combining the inner and outer housings together in a rotatable sub-assembly.

In preferred embodiments, the capping and cleansing devices of the invention are also manufactured to include a removable lid or seal attached to the outer housing to seal the device, thus separating the interior spaces and structures of the inner and outer housings from the external environment. Such a lid or seal prevents exposure of the device's interior, including the inner housing and compressible cleansing matrix, to the environment until the removable (preferably, peelable) lid or seal is removed, typically by a healthcare worker just prior to her/his use of the capping and cleansing device to clean, cleanse, or disinfect a needlefree connector to which a fluid connection is to be made.

In some preferred embodiments, the devices of the invention are sealed individually, while in other embodiments, 2-20 or more devices are sealed onto a single piece of lidding or sealing stock, after which they may be separated into individual sealed products or maintained in strip form, as a strip format having multiple devices all sealed to a single strip is a convenient format for use in healthcare environments, where such strips can be hung, for example, from an IV pole at a patient's bedside. Of course, the invention also includes embodiments wherein individually sealed capping and cleansing devices of the invention are provided in a format that includes multiple capping and cleansing devices. After sealing and packaging, the devices of the invention are sterilized using any suitable sterilization method (e.g., gamma or e-beam irradiation, treatment with ethylene oxide, etc.) compatible with the materials used to manufacture the particular device(s) of the invention. If desired, labeling information, logos, artwork, manufacturing, and/or regulatory data (e.g., lot number, expiration or “use by” dates, etc.) may also be printed or otherwise applied to individual capping and cleansing devices. In addition, information such as a bar code (to allow use of the device to tracked, for example) may also be included on individual capping and cleansing devices.

In preferred embodiments, such cleaning, cleansing, or disinfection of a vascular access connector (e.g., an NFC, male luer, or open female luer) substantially disrupts any microbial contamination, for example, microbial biofilm or other microbial contamination that may exist on surfaces contacted by the compressible cleansing matrix. If desired, the capping and cleansing device can be left in place (typically after cleansing desired region of the threaded vascular access connector attached thereto) in order to cap the connector until it is further accessed, thereby minimizing exposure of capped exterior surfaces of the threaded vascular access connector to potential pathogen contamination (and biofilm formation) from the surrounding environment. Lids or seals are typically installed during manufacture of a capping and cleansing device of the invention. In those embodiments where the capping and cleansing devices are sterilized during manufacture (e.g., by irradiation, exposure to ethylene oxide, etc.), lids or seals are preferably applied prior to packaging and sterilization.

Other aspects of the invention concern active methods of cleansing and/or capping vascular access connectors using a capping and cleansing device according to the invention. Such methods typically involve disengaging the engaging elements of the outer housing and inner housing after it has been connected to a needlefree connector, thus allowing a user to actively rotate or spin the outer housing in relation to the inner housing and needlefree connector to which the device of the invention is secured. Such disengagement does not impair contact between the device's compressible cleansing matrix and the associated surface(s) of the needlefree connector. Spinning or rotation of the outer housing in relation to the inner housing, and the associated surface(s) of the vascular access connector, allow those surfaces to be actively scrubbed, thereby cleansing them. Preferably, such active cleansing methods provide for the disruption of any biofilm present on the surface(s) of the needlefree connector associated the capping and cleansing device. And in those embodiments where the compressible cleansing matrix contains one or more antimicrobial reagents, microbes and pathogens present in such biofilm and/or on such surface(s) are preferably destroyed or rendered nonviable.

Thus, a related aspect of the invention concerns methods of using the single-use capping and cleansing devices of the invention. Such methods include using the devices to cleanse and, if desired, cap vascular access connectors (e.g., NFCs, male luers, open female luers). To perform such methods, the portion of a vascular access connector to be cleansed (and, in many embodiments, capped) is threaded into the central bore of the inner housing of a capping and cleansing device, typically after the user (e.g., a nurse) removes a seal that spans the opening to the main cavity in the device. Such insertion brings the site of the vascular access connector into contact with (i.e., brought into cleansing association with) the compressible cleansing matrix portion(s) of the device. In preferred practice, once the compressible cleansing matrix is in contact with the surface(s) of the vascular access connector to be cleansed, the outer housing automatically disengages the engaging element(s) in the inner and outer housings to allow rotation of the outer housing (and associated compressible cleansing matrix) in relation to the inner housing and needlefree connector previously releasably connected to capping and cleansing device. Such contact and cleansing action can be for any desired period, with periods of about one second to about ten to twenty seconds being particularly preferred. After cleansing, the capping and cleansing device can be removed from the vascular access connector, after which the capping and cleansing device may be discarded. Alternatively, after cleansing, the capping and cleansing device can be left attached to the needlefree connector, capping a portion thereof until such time as access to the needlefree connector is desired, capping it and protecting it from contamination. At that time, the capping and cleansing device can be removed and discarded. If desired, just prior to removal, a cleansing process can be repeated.

After removal of a capping and cleansing device from a cleansed vascular access connector, a fluid-containing medical reservoir (e.g., a syringe containing a medication, an IV bag, etc.) may be immediately connected to the cleansed vascular access connector. In preferred embodiments where the cleansing reagent is a solution, the surface(s) of the needlefree connector is preferably allowed to dry (or is(are) dried, for example, by wiping with a sterile, absorbent cloth or wipe, which cloth or wipe may be dry or wetted with a volatile, compatible solution such as 70-100% alcohol) prior to connecting the needlefree connector to a fluid reservoir. In preferred practice, such cleansing methods result in at least a 2-fold, 5-fold, or 10-fold or more reduction in microorganism contamination on the accessible surface(s) that have been cleansed. Even more preferably, the level of reduction may exceed a 100-fold, a 10³-fold, a 10⁴-fold, a 10⁵-fold, a 10⁶-fold, or 10⁷-fold reduction in microorganism contamination on the accessible fitting surface.

In addition to methods for cleansing accessible surfaces of vascular access devices and the like, the devices of the invention provide methods of reducing infection risk in a patient connected to devices, such as a peripheral IV line, a central IV line, a peripherally inserted central catheter, hemodialysis catheter, or other fluid line configured for pumped or gravity-fed delivery of fluids directly into the patient's vasculature. The risk reduction afforded by the devices of the invention may vary depending upon many factors, such as patient age and condition, the condition being treated, the location where medical services are being delivered, patient density, the level of contaminating microorganisms in the environment, the quality of air handling equipment in the medical facility, the degree of training of medical personnel charged with cleansing the access device, the method(s) used to periodically cleanse the vascular access device, intervals between cleansing procedures, the particular configuration of the capping and cleansing device, the particular configuration of the vascular access connector, whether the capping and cleansing device is left on the cleansed site of the vascular access connector in order to provide capping, etc. Risk reduction can be established using any suitable method, for example, by assessing HAI frequency in the presence and absence of using cleansing devices according to the invention. Reductions of HAI infection risk of 1-100% or more, including up to 1000% or more, are envisioned through use of capping and cleansing devices according to the invention. As will be appreciated, reductions in infection risk (e.g., HAI risk) will translate to improved patient outcomes (through reduced morbidity and mortality) and reduced expenditure on treating HAI's.

### Representative Embodiments

To further illustrate and describe certain preferred, representative embodiments of the invention, the reader is directed to the appended drawings, FIGS. 1 and 2. A description of these preferred, representative embodiments follows.

FIG. 1 shows an exploded cutaway view of a representative example of a capping and cleansing device of the invention (10) designed to be threaded onto a threaded open female luer connector (100), as well as a cutaway view of such a device (10) threaded onto such a connector (100). As shown in FIG. 1, the capping and cleansing device comprises an inner housing (12), an outer housing (30), and a compressible cleansing matrix (50; here, made from medical grade foam). The inner housing has a central cylindrical bore (14) that extends completely through the housing, with the bore having a first opening (16) at the top of the housing and second opening (18) at the bottom of the inner housing. To provide for threaded engagement of and securement to a threaded open female luer connector, the inner surface of the cylindrical bore (14) includes threads (20) complementary to the thread tabs (102) on the outer surface of the upper region (101) of the open female luer connector (100). The threaded open female luer connector (100) includes an open cavity (104) whose inner surface (106) is often exposed to the environment and can thus become contaminated with potentially pathogenic microorganisms. The compressible cleansing matrix (50) is designed to contact at least a portion of the inner surface (106) of the open female luer cavity (104). Preferably, the compressible cleansing matrix (50) contacts substantially all of inner surfaces (106) of the female luer cavity (104), including those on the sidewall (108) and bottom (110). Typically, a fluid opening (not shown) is centrally disposed at the bottom (110) of the female luer cavity, which opening provides fluid access to the flow path into and through the threaded female luer connector (100). In preferred embodiments, the compressible cleansing matrix (50b) includes a self-centering, tapered elastomeric pin (70; or similar structure) that seats in the fluid opening at the bottom (110) of the female luer cavity to prevent entry of fluid, particulates, etc. into the flow path of threaded female luer connector (100) while a capping and cleansing device of the invention (10) is connected thereto. The pin (60) can be made of any suitable material, and is preferably inserted into a recess formed or made in the lower portion of the compressible cleansing matrix (50).

The outer housing (30) has a cylindrical sidewall (32) that forms a cavity (34) designed to accept and rotatably retain the inner housing (12) when the inner (12) and outer (30) housings are assembled during manufacturing. Here, rotatable retention is provided by a series of tapered projections (38) that project from the inner surface (37) of the outer housing's sidewall (32) into the outer housing's cavity (34). These projections (38) form a seat or inner housing engaging region (39) designed to extend under the flange region (20) of the inner housing (12) when the inner and outer (12, 30) housings are assembled, allowing the inner housing (12) to be rotatably retained in the outer housing's main cavity (34). In this embodiment, the outer housing (30) also includes a matrix well or upper cavity (40) configured to accept a compressible cleansing matrix (50) and impart rotational force from the outer housing to the compressible cleansing matrix (50). Here, the inner surface (42) of the matrix well includes a plurality of vertical tabs (44) that project into the matrix well (40) to engage the compressible cleansing matrix (50) more effectively when it is assembled into the matrix well (40). In this way, the outer housing (30) and the cleansing matrix (50) can rotate independently of the inner housing (12) after the capping and cleansing device (10) has been threaded onto a threaded open female luer connector (100). In this embodiment, when the outer housing (30) is squeezed, for example, between a nurse's fingers, the outer housing deforms sufficiently so as to allow several of the tapered projections (38) and or other regions of the inner surface (37) of the outer housing (30) to contact opposing surfaces on the outer surface of the inner housing (12).

In the embodiment depicted in FIG. 1, the region of the inner surface (37) of the outer housing (30) opposite the flange region (20) of the inner housing (12) can, when a healthcare worker squeezes the outer housing (30) with sufficient force to, for example, unthread the cap (10) from the connector (100), engage the opposing surface of the flange region (20) of the inner housing (12), allowing the cap (10) to be unscrewed (or unthreaded) from the open female luer connector (100; e.g., a connector on a central line). In other words, when sufficient force is applied, the outer (30) and inner (12) housings can then be made rotate together, allowing the capping and cleansing device (10) to be removed from the connector (100). Attachment of the capping and cleansing device (10) to be removed from the connector (100) is also accomplished in the same way, albeit by rotating the device (10) in the opposite direction to screw (thread) it onto the connector (100).

The representative embodiment shown in FIG. 1 also includes a seal (60) built into the outer housing (30) to engage a sealing surface (22) on a tapered portion (24) above the flange region (20) of the inner housing (20). Together, the seal and sealing surface (60, 22) work to limit loss of the liquid disinfectant when the cap (10) is left to dwell on the connector (100) after attachment to provide passive protection and disinfection to the inner surfaces (108, 110) of the connector (100). In preferred embodiments, the cap (10) can be left on the connector (100) is “passive” mode for up to 7 days.

The representative cap embodiment shown in FIG. 1 also includes a peelable seal or lid (80) that is attached via its upper surface (82) during manufacturing to the lower surface of the outer housing (30) at the perimeter (35) of the main cavity in order seal the device to prevent evaporation of the liquid disinfectant and maintain sterility following sterilization. Such attachment can be by anu suitable method, including heat sealing. As those in the art will appreciate, the particular method used will depend on such factors as the lidding (80) stock, the material used to form the outer housing (30), the liquid disinfectant, etc.

FIG. 2 show a top view and an exploded cross-section view of a representative example of a capping and cleansing device of the invention designed to be threaded onto a threaded male luer connector (200), as well as a cross-section view (A-A) of such a device (300) threaded onto such a connector (200). As shown in FIG. 2, the capping and cleansing device (300) comprises an inner housing (310), an outer housing (350), and a compressible cleansing matrix (380; here, made from medical grade foam). The inner housing (310) has a central smooth cylindrical bore (312) that extends completely through the housing, with the bore having a first opening (314) at the top of the housing and second opening (316) at the bottom of the inner housing. To provide for threaded engagement of and securement to a threaded male luer connector (200), the outer surface (318) of the lower section (320) of the inner housing (310) includes thread tabs (322) designed to engage the threaded region (202) of the inner surface (204) of the male luer connector (200). The male luer connector (200) includes a tapered central portion (206) having a bore (208) through which fluid flows. The tip (210) of the tapered central portion (206) includes a fluid path opening (211). The outer surface (207) of the tapered central portion (206) is typically smooth and is often exposed to the environment and can thus become contaminated with potentially pathogenic microorganisms.

The capping and cleansing device (300) includes a compressible cleansing matrix (380) designed to contact at least a portion of the smooth tapered central portion (206) the male connector (200) and seal its fluid inlet (211). The compressible cleansing matrix (380) can be formed from any suitable material, including a medical grade foam. FIG. 2 shows two different configurations (380a and 380b) for compressible cleansing matrix (380). In each of these embodiments, the upper portion (382) of compressible cleansing matrix (380) is configured to engage and be retained in a matrix well (390) of the outer housing (350). Retaining ribs (391) or similar structures are advantageously positioned on the inner surface of the matrix well (380). The lower portion (383) of the compressible cleansing matrix (380) includes a cleansing cavity (384) adapted to receive the tip (210) of the male luer taper (206) such the smooth outer surface of the tip of the male luer taper comes into contact with the inner surface (385) of the cleansing cavity (384). Preferably, the compressible cleansing matrix (380) contacts much if not all of the smooth outer surface of the tip of the male luer taper protected by the device (300) of the invention.

It is also desired that the compressible cleansing matrix (380) also include a structure to seal the fluid opening (211) of the male luer connector when the device (300) is securely attached the connector (200). Any suitable approach can be taken in this regard. FIG. 2 shows two such configurations. For example, the cleansing cavity (384) of the compressible cleansing matrix (380b) can be formed to include a central protrusion (386) extending into the cleansing cavity (384) from its upper surface. In another embodiment, of the compressible cleansing matrix (380a), the compressible cleansing matrix (380) includes a central sealing pin (390) that extends into the cleansing cavity (384). The sealing pin can be made from any suitable material, including a plastic or other elastomer.

The compressible cleansing matrix (380) is preferably designed to extend into the central smooth cylindrical bore (312) of the inner housing (310) so that it not only engages the outer surface of the male luer taper and seals its fluid inlet (211) but also engages the threaded region of the male luer connector above the area where the complementary threads of the inner and outer housings engage to secure the device (300) to the connector (200).

The outer housing (350) has a cylindrical sidewall (351) that forms a main cavity (352) designed to accept and rotatably retain the inner housing (310) when the inner (310) and outer (350) housings are assembled during manufacturing. Here, rotatable retention is provided by a series of tapered projections (355) that project from the inner surface of the outer housing's sidewall (356) into the outer housing's main cavity (352). These projections (355) form a seat or inner housing engaging region designed to extend under the flange region (325) of the inner housing (310) when the inner and outer (310, 350) housings are assembled, allowing the inner housing (310) to be rotatably retained in the outer housing's main cavity (352). In this embodiment, the outer housing (350) also includes a matrix well or upper cavity (390) configured to accept a compressible cleansing matrix (380) and impart rotational force from the outer housing to the compressible cleansing matrix (380), particularly during a cleansing operation. Here, the inner surface of the matrix well (390) includes a plurality of vertical tabs (391) that project into the matrix well (390) to engage the compressible cleansing matrix (380) more effectively when it is assembled into the matrix well (390). In this way, the outer housing (350) and the cleansing matrix (3800) can rotate independently of the inner housing (310) after the capping and cleansing device (300) has been threaded onto a threaded male luer connector (200) having a central male luer taper (206). In this embodiment, when the outer housing (350) is squeezed, for example, between a nurse's fingers, the outer housing deforms sufficiently so as to allow the outer housing (350) to contact opposing surfaces on the outer surface of the inner housing (310), locking them together so that they can be rotated in unison in order to attach or detach the capping and cleansing device (300) from the connector (200).

In the embodiment depicted in FIG. 2, the region of the inner surface (356) of the outer housing (350) opposite the flange region (325) of the inner housing (310) can, when a healthcare worker squeezes the outer housing (350) with sufficient force to, for example, unthread the cap (300) from the connector (200), engage the opposing surface of the flange region (325) of the inner housing (310), allowing the cap (300) to be unscrewed (or unthreaded) from the male luer connector (200). In other words, when sufficient force is applied, the outer (350) and inner (310) housings can then be made rotate together, allowing the capping and cleansing device (300) to be removed from the connector (200). Attachment of the capping and cleansing device (300) to be removed from the connector (200) is also accomplished in the same way, albeit by rotating the device (300) in the opposite direction to screw (thread) it onto the connector (200).

The representative embodiment shown in FIG. 2 also includes a seal (400) built into the outer housing (300) to engage a sealing surface (326) on a tapered portion (327) above the flange region (325) of the inner housing (310). Together, the seal and sealing surface (400, 326) work to limit loss of the liquid disinfectant when the cap (300) is left to dwell on the connector (200) after attachment to provide passive protection and disinfection to target surfaces of the connector (200). In preferred embodiments, the cap (300) can be left on the connector (200) is “passive” mode for up to 7 days.

The representative cap embodiment shown in FIG. 2 also includes a peelable seal or lid (80) that is attached via its upper surface (82) during manufacturing to the lower surface of the outer housing (350) at the perimeter (359) of the main cavity (390) in order seal the device to prevent evaporation of the liquid disinfectant and maintain sterility following sterilization. Such attachment can be by anu suitable method, including heat sealing. As those in the art will appreciate, the particular method used will depend on such factors as the lidding (80) stock, the material used to form the outer housing (300), the liquid disinfectant, etc.

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law.

What is claimed is:

1. A capping and cleansing device for a threaded vascular access connector, the capping and cleansing device comprising:
(a) an inner housing rotatably retained in an outer housing, which inner housing comprises (i) a sidewall that bounds a central bore and (ii) threads or thread tabs configured to engage a complementary threaded region of the threaded vascular access connector so to allow the capping and cleansing device to be screwed onto and unscrewed from the threaded region of the threaded vascular access connector, wherein a compressible cleansing matrix attached to the outer housing protrudes into the central bore to allow contact with one or more exterior surfaces of the threaded vascular access connector when the capping and cleansing device is screwed onto the threaded region of the threaded vascular access connector;
(b) the outer housing, which outer housing comprises a cavity in which the inner housing is rotatably retained, wherein the outer housing is configured to releasably engage the inner housing so as to allow the outer housing to (i) independently rotate in one or both directions in relation to the inner housing when the outer housing is not releasably engaging the inner housing and (ii) rotate in unison with the inner housing when the outer housing releasably engages the inner housing; and
(c) the compressible cleansing matrix associated with the outer housing and protruding into the cavity of the outer housing and into the central bore of the inner housing, which compressible cleansing matrix rotates with the outer housing, which compressible cleansing matrix is impregnated with a liquid disinfectant.

2. A capping and cleansing device according to claim 1 wherein the threaded vascular access connector is a threaded valve portion of a needlefree connector, a threaded male luer connector, and a threaded open female luer connector.

3. A capping and cleansing device according to claim 1 wherein the threads or thread tabs of the inner housing are disposed on (i) a portion of a bore-facing surface of the central bore or (ii) a portion of an outer surface of the inner housing.

4. A capping and cleansing device according to claim 1 that further comprises a removable seal to seal an interior of the device from the external environment.

5. A capping and cleansing device according to claim 1, wherein the cleansing reagent comprises isopropyl alcohol, optionally a 70% isopropyl alcohol solution.

6. A capping and cleansing device according to claim 1, wherein the outer housing comprises an outer surface having a plurality of vertical ridges.

7. A method of cleansing a threaded vascular access connector, comprising:
(a) connecting a threaded vascular access connector to a capping and cleansing device according to claim 1 such that the one or more surfaces of the connector engage and at least partially compress the compressible cleansing matrix of the capping and cleansing device; and
(b) rotating the outer housing of the capping and cleansing device in relation to the inner housing of the device, thereby cleansing one or more exterior surface(s) of the connector contacted by the compressible cleansing matrix.

8. A method according to claim 7 that further comprises leaving the capping and cleansing device connected to the threaded vascular access connector after cleansing, thereby capping the connector.
